SEQUENCE LISTING (13 sequences)

SEQ ID NO: 1 - DNA, 3897 bp, Artificial Sequence
  Synthesized Construct
atgcagatctttgtgaagaccctgaacggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtgctgcgcctgcgcggcgtcgtaagcggtgaattcatgtacccatacgacgtcccagactacgctatgaacaacttccaggccatcctgactcaggtgagaatgctgctctccagccatcagcccagcctggtgcaggccctcttggacaacctgctgaaggaggacctcctctccagggaataccactgcactctgctccatgagcctgatagtgaggctctggccaggaagatctctttgaccctactagagaaaggagacctggatttggccctcctggggtgggcccggagtgggctgcagcccccagcagccgagaggggccccggccacagtgaccatggtggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacgccttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgcttgagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcggcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 2 - protein, 1298 aa, Artificial Sequence
  Synthesized Construct
MQIFVKTLTGKTITLEVEPSDTIENVKAKIQDKEGIPPDQQRLIFAGKQLEDGRTLSDYNIQKESTLHLVLRLRGVVSGEFMYPYDVPDYAMNNFQAILTQVRMLLSSHQPSLVQALLDNLLKEDLLSREYHCTLLHEPDSEALARKISLTLLEKGDLDLALLGWARSGLQPPAAERGPGHSDHGGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDAFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLRQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 3 - DNA, 225 bp, Homo sapiens
atgcagatctttgtgaagaccctgaacggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtgctgcgcctgcgcggc

SEQ ID NO: 4 - protein, 75 aa, Homo sapiens
MQIFVKTLTGKTITLEVEPSDTIENVKAKIQDKEGIPPDQQRLIFAGKQLEDGRTLSDYNIQKESTLHLVLRLRG

SEQ ID NO: 5 - DNA, 3690 bp, Homo sapiens
atgctgggtgagcggagattccaggcactggccagggcagctgccctgactccaagggctgccatgaacaacttccaggccatcctgactcaggtgagaatgctgctctccagccatcagcccagcctggtgcaggccctcttggacaacctgctgaaggaggacctcctctccagggaataccactgcactctgctccatgagcctgatagtgaggctctggccaggaagatctctttgaccctactagagaaaggagacctggatttggccctcctggggtgggcccggagtgggctgcagcccccagcagccgagaggggccccggccacagtgaccatggtggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcatagagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgagaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 6 - protein, 1229 aa, Homo sapiens
MLGERRFQALARAAALTPRAAMNNFQAILTQVRMLLSSHQPSLVQALLDNLLKEDLLSREYHCTLLHEPDSEALARKISLTLLEKGDLDLALLGWARSGLQPPAAERGPGHSDHGGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFIEHIGPDEVIGESMEMPAEVGQKSQKRPFPEELPADLKHWKPAEPPTVVTGSLLVRPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 7 - DNA, 867 bp, Homo sapiens
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 8 - protein, 287 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVLALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 9 - DNA, 990 bp, Homo sapiens
atggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaa

SEQ ID NO: 10 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 11 - protein, 31 aa, Artificial Sequence
  Synthesized Construct
PTSPDRPGSTSPFAPSATDLPSMPEPALTSR

SEQ ID NO: 12 - protein, 6 aa, Artificial Sequence
  Synthesized Construct
VVSGEF

SEQ ID NO: 13 - protein, 10 aa, Artificial Sequence
  Synthesized Construct
MYPYDVPDYA

(19) United States
(12) Patent Application Publication
(10) Pub. No.: US 2009/0047262 A1
(43) Pub. Date: Feb. 19, 2009

(54) Expression of class II transactivator fusion proteins for control of tumor growth

(75) Inventors: Gorazd Drozina, San Francisco, CA (US); B. Matija Peterlin, San Francisco, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/981,199

(22) Filed: Oct. 30, 2007

Related U.S. Application Data
(60) Provisional application No. 60/856,587, filed on Nov. 2, 2006.

(51) Int. Cl. A61K 35/12; C12N 15/11; C12N 15/00; A61P 31/00; C12N 5/06

(52) U.S. Cl. 424/93.21; 536/23.4; 435/320.1; 435/325

(57) ABSTRACT

The present invention relates to tumor immunotherapy. In particular, the present invention provides methods and compositions for converting cancer cells into antigen presenting cells. Thus the present invention provides immunogenic compositions for the treatment and prevention of cancer.

This application claims the benefit of U.S. Provisional Application No. 60/856,587, filed on Nov. 2, 2006, herein incorporated by reference in its entirety.

This invention was made with Government support under Grant Number RO1 AI 050770 awarded by the National Institutes of Health. The Government has certain rights in this invention.

### FIELD OF THE INVENTION

The present invention relates to tumor immunotherapy. In particular, the present invention provides methods and compositions for converting cancer cells into antigen presenting cells. Thus the present invention provides immunogenic compositions for the treatment and prevention of cancer.

### BACKGROUND OF THE INVENTION

According to the American Cancer Society, approximately 1.4 million new cases of cancer will be diagnosed in the United States in 2005, and more than 550,000 people will die of the disease. In fact, cancer is the second leading cause of death in the United States, behind heart disease. Although improvements have been made in cancer detection, diagnosis, and treatment, for many types of cancer, nearly 35 percent of patients diagnosed with cancer will die within five years of diagnosis. Pancreatic cancer is particularly lethal, ranking below lung cancer, colon cancer and breast cancer, as the leading cause of cancer death in the United States. The prognosis is poor, with a five-year survival rate of about four percent, and a median survival time post-diagnosis of around six months. Early detection of pancreatic cancer is difficult, and most patients are found to have inoperable disease (Freelove and Walling, Am Fam Physician, 73:485-492, 2006), due to metastasis and invasion of major vessels surrounding the pancreas. For locally advanced, unresectable, and metastatic disease, treatment is primarily palliative, although chemotherapy and radiation therapy may also be offered.

The most frequently employed chemotherapies for pancreatic cancer are the fluoropyrimidines (Sun and Haller, “Pancreatic, Gastric, and Other Gastrointestinal Cancers,” in Scientific American Medicine, Chapter 12, pp. 1-22, 2004), 5-fluorouracil and 2′,2′-difluorodeoxycytidine (gemcitabine hydrochloride or GEMZAR). These drugs induce apoptosis of cancer cells by inhibiting DNA replication. Frequent side effects of these medications include low blood counts, nausea and vomiting. In addition, although chemoradiation treatment regimens permit a small minority of patients to become surgical candidates, chemotherapy generally only extends the survival time of pancreatic cancer patients by about six months.

Thus, more effective therapy regimens for treating cancer are needed. In particular, it would be desirable to have alternative treatment options for aggressive malignancies such as pancreatic cancer.

### SUMMARY OF THE INVENTION

The present invention relates to tumor immunotherapy. In particular, the present invention provides methods and compositions for converting cancer cells into antigen presenting cells. Thus the present invention provides immunogenic compositions for the treatment and prevention of cancer.

The present invention provides an isolated nucleic acid encoding a mammalian ubiquitin/major histocompatibility complex class II transactivator isoform 1 (Ub.CIITA1) fusion protein. In some embodiments, the Ub.CIITA1 fusion protein is the protein set forth in SEQ ID NO:2 or a transcriptionally active variant that differs from SEQ ID NO:2 by less than 1%. In some preferred embodiments, the Ub.CIITA1 fusion protein comprises one or both of a K48R substitution and a K63R substitution. In some embodiments, the nucleic acid is set forth in SEQ ID NO:1. Also provided are expression vectors comprising a nucleic acid encoding a mammalian ubiquitin/major histocompatibility complex class II transactivator isoform 1 (Ub.CIITA1) fusion protein. In some preferred embodiments, the vector is a recombinant retrovirus, which in particularly preferred embodiments further comprises a selection marker. Also provided are isolated host cells comprising an expression vector comprising a nucleic acid encoding a mammalian ubiquitin/major histocompatibility complex class II transactivator isoform 1 (Ub.CIITA1) fusion protein. In preferred embodiments, the host cell is a cancer cell. In particularly preferred embodiments, the host cell further comprises an expression vector comprising a nucleic acid encoding a mammalian co-stimulatory molecule. In some embodiments, the co-stimulatory molecule comprises one or both of CD80 (B7.1) and CD86 (B7.2). In preferred embodiments, the CD80 (B7.1) is the protein set forth in SEQ ID NO:8 or a biologically active variant that differs from SEQ ID NO:8 by less than 1%, and/or the CD86 (B7.2) is the protein set forth in SEQ ID NO:10 or a biologically active variant that differs from SEQ ID NO:10 by less than 1%. In some preferred embodiments, the CD80 (B7.1) is encoded by the nucleic acid set forth in SEQ ID NO:7 and/or the CD86 (B7.2) is encoded by the nucleic acid set forth in SEQ ID NO:9.

Additionally, the present invention provides methods for inducing an immune response, comprising: contacting a tumor cell with an expression vector comprising a nucleic acid encoding a mammalian ubiquitin/major histocompatibility complex class II transactivator isoform 1 (Ub.CIITA1) fusion protein, under conditions suitable for producing transfected tumor cells expressing MHC class II molecules; and administering the transfected tumor cells to a subject under conditions suitable for inducing an immune response against the transfected tumor cells. In some preferred embodiments, the expression vector further comprises a nucleic acid encoding a mammalian co-stimulatory molecule and the transfected tumor cells further express costimulatory molecules. In a subset of these embodiments, the expression vector comprises at least two replication deficient retrovirus vectors. In some particularly preferred embodiments, the tumor cell is obtained from a biopsy of a tumor from the subject. In some embodiments the tumor is from an organ selected from but not limited to breast, pancreas, gall bladder, stomach and liver. In some embodiments, the methods further comprise the step of selecting the transfected tumor cells for surface expression of one or both of MHC class II and costimulatory molecules by cell sorting prior to the administering step. The present invention also provides embodiments, which further comprise the step of irradiating the transfected tumor cells to prior to the administering step. In some embodiments, the administering is by injection of a femoral vein of the subject. In some embodiments, the methods further comprise the step of treating the subject with an adjunct immunomodulatory agent after the administering step. In a subset of these embodiments, the adjunct immunomodulatory agent is selected from the group consisting of IL-2 and anti-CD25 antibody. In some preferred embodiments, the immune response comprises a transfected tumor cell-reactive proliferative response by lymphocytes from the subject. In particularly preferred embodiments, the immune response comprises shrinking an existing tumor of the subject and/or delaying development of tumor metastases in the subject.

Moreover, the present invention provides methods for inducing an immune response, comprising administering transfected tumor cells expressing MHC class II molecules to a subject under conditions suitable for inducing an immune response against the transfected tumor cells, wherein the transfected tumor cells comprise an expression vector comprising a nucleic acid encoding a mammalian ubiquitin/major histocompatibility complex class II transactivator isoform 1 (Ub.CIITA1) fusion protein. In further embodiments, the expression vector further comprises a nucleic acid encoding a mammalian co-stimulatory molecule and the transfected tumor cells further express costimulatory molecules. In a subset of these embodiments, the expression vector comprises at least two replication deficient retrovirus vectors. In some particularly preferred embodiments, the tumor cell is obtained from a biopsy of a tumor from the subject. In some embodiments the tumor is from an organ selected from but not limited to breast, pancreas, gall bladder, stomach and liver. In some embodiments, the methods further comprise the step of selecting the transfected tumor cells for surface expression of one or both of MHC class II and costimulatory molecules by cell sorting prior to the administering step. The present invention also provides embodiments, which further comprise the step of irradiating the transfected tumor cells to prior to the administering step. In some embodiments, the administering is by injection of a femoral vein of the subject. In some embodiments, the methods further comprise the step of treating the subject with an adjunct immunomodulatory agent after the administering step. In a subset of these embodiments, the adjunct immunomodulatory agent is selected from the group consisting of IL-2 and anti-CD25 antibody. In some preferred embodiments, the immune response comprises a transfected tumor cell-reactive proliferative response by lymphocytes from the subject. In particularly preferred embodiments, the immune response comprises shrinking an existing tumor of the subject and/or delaying development of tumor metastases in the subject.

The present invention also provides kits for inducing an immune response, comprising: i) an expression vector comprising a nucleic acid encoding a mammalian ubiquitin/major histocompatibility complex class II transactivator isoform 1 (Ub.CIITA1) fusion protein; ii) instructions for contacting a tumor cell with the expression vector to produce transfected tumor cells expressing MHC class II molecules; and iii) instructions for administering the transfected tumor cells to a subject to induce an immune response against the transfected tumor cells. In some embodiments the expression vector further comprises a nucleic acid encoding a mammalian co-stimulatory molecule. In some preferred embodiments, the expression vector comprises at least two replication deficient retrovirus vectors. In some particularly preferred embodiments, the tumor cell is obtained from a biopsy of a tumor from the subject.

### DESCRIPTION OF THE DRAWINGS

FIG. 1A shows the cDNA (SEQ ID NO:1) sequence and FIG. 1B shows the amino acid (SEQ ID NO:2) sequence of an exemplary ubiquitin/class II transactivator isoform 1 (Ub.CIITA IF1) fusion of the present invention. The nucleotide sequence corresponding to an HA tag and a linker is shown in capital letters. The amino acid sequence of the HA tag is VVSGEF (SEQ ID NO:12) and the amino acid sequence of the linker is MYPYDVPDYA (SEQ ID NO:13). In some preferred embodiments, the Ub.CIITA1 fusion protein comprises one or both of a K48R substitution and a K63R substitution, which prevent ubiquitin polymerization and degradation.

FIG. 2A shows the cDNA (SEQ ID NO:3) sequence and FIG. 2B shows the amino acid (SEQ ID NO:4) sequence of a human ubiquitin (Ub) monomer.

FIG. 3A shows the cDNA (SEQ ID NO:5) sequence and FIG. 3B shows the amino acid (SEQ ID NO:6) sequence of human class II transactivator isoform 1 (CIITA IF1).

FIG. 4A shows the cDNA (SEQ ID NO:7) sequence and FIG. 4B shows the amino acid (SEQ ID NO:8) sequence of human CD80 (B7.1).

FIG. 5A shows the cDNA (SEQ ID NO:9) sequence and FIG. 5B shows the amino acid (SEQ ID NO:10) sequence of human CD86 (B7.2).

FIG. 6 illustrates that an ubiquitylated CIITA IF1 protein is more active than its counterpart that is not ubiquitylated. FIG. 6A illustrates that the h:Ub.CIITA1 chimera is expressed at lower levels than the wild type f:CIITA1 protein: f:CIITA1 (lane 1) and h:Ub.CIITA1 fusion proteins (lane 2) expressed in COS cells. Arrows on the right indicate the presence of f:CIITA1 and the h:Ub.CIITA1 fusion proteins, respectively. FIG. 6B shows that the degradation of the h:Ub.CIITA1 chimera is rapid. The h:Ub.CIITA1 chimera was expressed in COS cells, which were starved for cysteine and methionine and then incubated in medium containing ³⁵S cysteine and ³⁵S methionine. Samples were collected at the indicated time points. The arrow on the left indicates the presence of the h:Ub.CIITA1 chimera. FIG. 6C shows that the proteasomal inhibitor ALLN increases levels of the h:Ub.CIITA1 chimera. The h:Ub.CIITA1 chimera (lanes 1 and 2) was expressed in COS cells. Before lysis, cells were treated with ALLN for 6 hours (lane 2) or with the solvent as the control (lane 1). Arrow on the right indicates the presence of the h:Ub.CIITA1 chimera. FIG. 6D shows that the h:Ub.CIITA1 chimera is transcriptionally more active than mutant f:CIITA1(S357A) protein. A pDRASCAT reporter was co-expressed in COS cells with mutant f:CIITA1(S357A) protein and the h:Ub.CIITA1 chimera. Fold transactivation represents the ratio between activities of the CIITA IF1 and the reporter plasmid alone. Error bars give standard errors of the mean for two experiments performed in duplicate. The panel to the left of the CAT data presents the input of mutant f:CIITA1(S357A) and the h:Ub.CIITA1 fusion proteins, respectively.

FIG. 7 provides a model of LPS signaling through TLR4. Briefly, LPS binds TLR4, which is in the complex with MD2 and CD14. The signaling cascade (1) continues via the adaptor molecules Myd88, IRAK1/4, and MEK1/2 and results in an activation of Erk1/2 kinase, which phosphorylates CIITA IF1 (2). This phosphorylation is followed by the ubiquitylation of CIITA IF1 (3) that is then positioned on the MHC II enhanceosome (depicted as RFX:RFX:NF—Y) on S, X and Y boxes of the CIITA promoter (depicted as S, X, Y). CIITA IF1 attracts not only general transcription factors that initiate transcription (4), but also the positive transcription elongation factor b (P-TEFb) composed of cyclin T1 (CycT1) and the cyclin-dependant kinase 9 (Cdk9). Cdk9 phosphorylates the C terminal domain (CTD) of RNA polymerase II (RNAPII) (depicted as arrows pointing from Cdk9 to the encircled P on the CTD), as well as N-TEF (not pictured) and facilitates the elongation of MHC class II transcription (5) leading to co-transcriptional processing of their mRNA species (depicted as bold arrow to the right in RNAPII). MHC class II determinants are assembled in the endoplasmic reticulum and travel to the trans-Golgi network (6). After the fusion with the late endosome, antigen processing begins (7) resulting in the presentation of antigenic peptides on the cell surface (8). Arrows point in the direction of these processes. The thin double line on the top represents cellular membranes and the thick line on the bottom represents DNA.

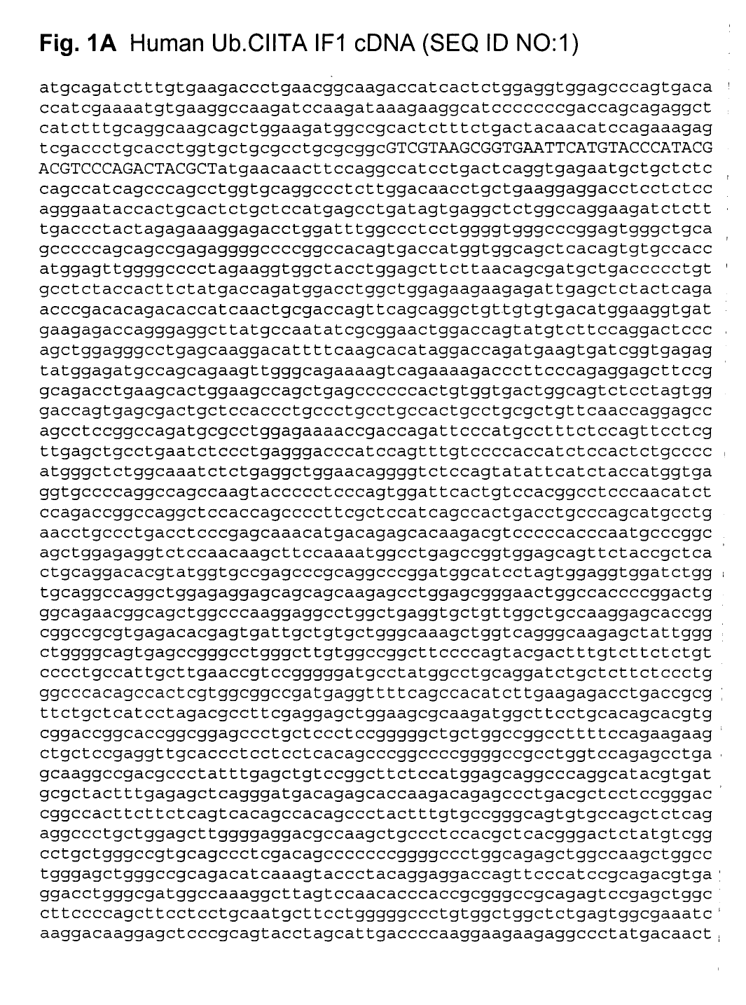

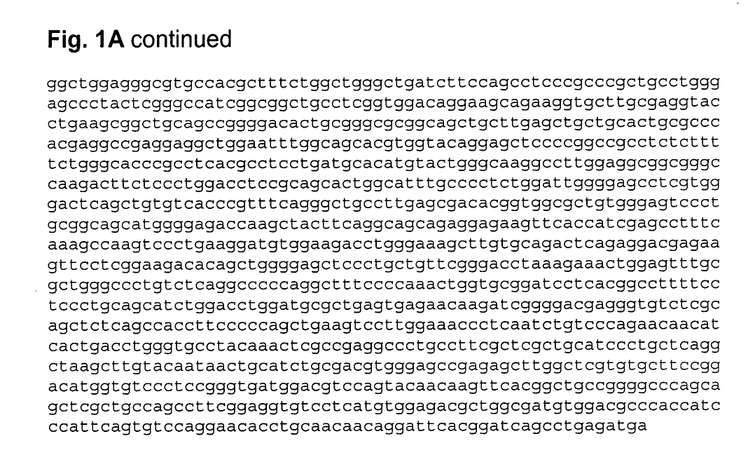

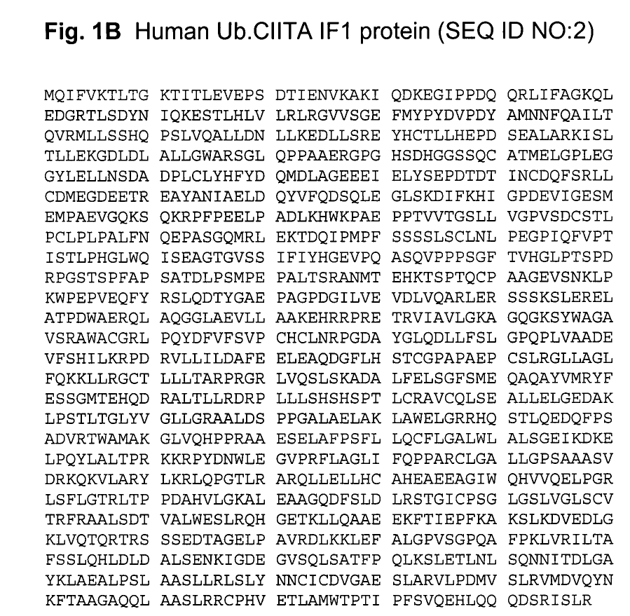

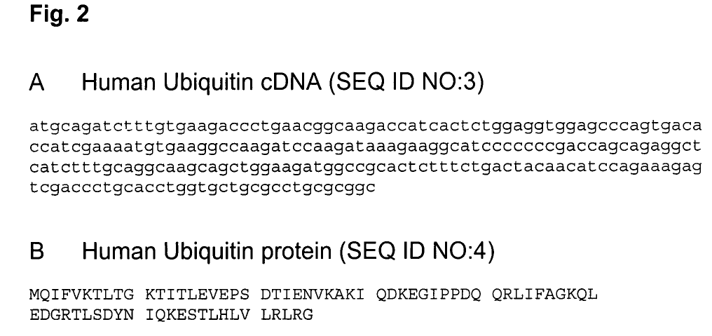

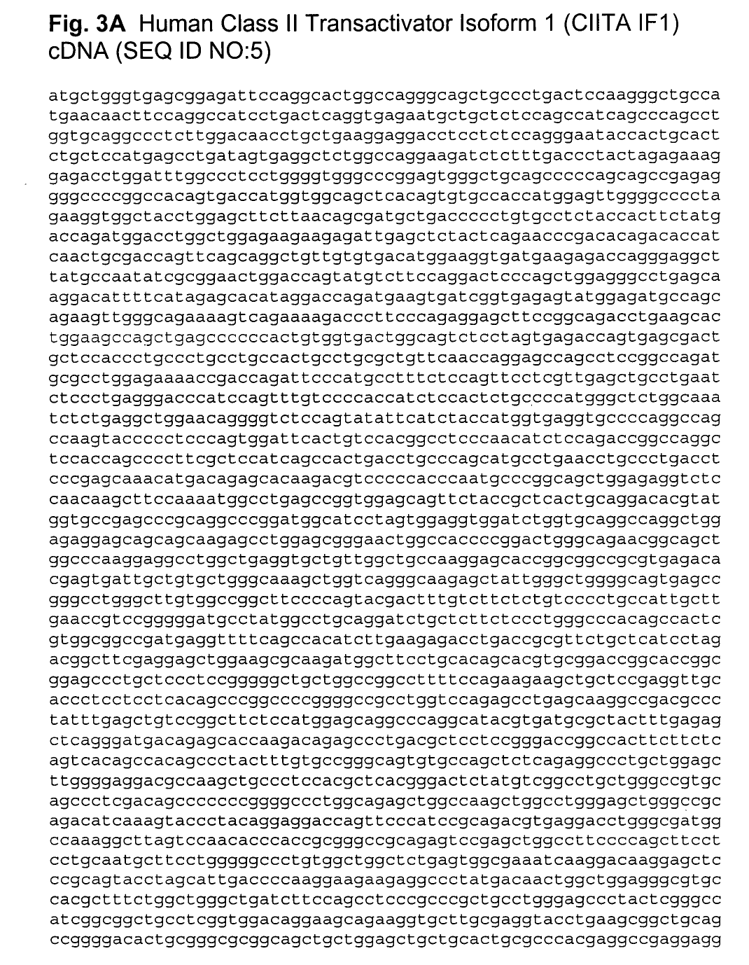

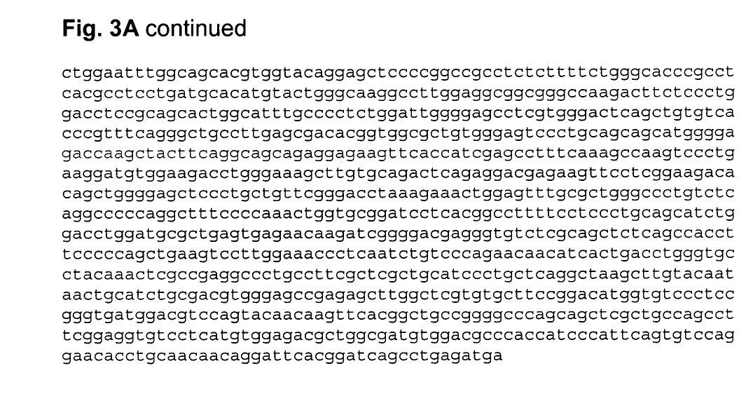

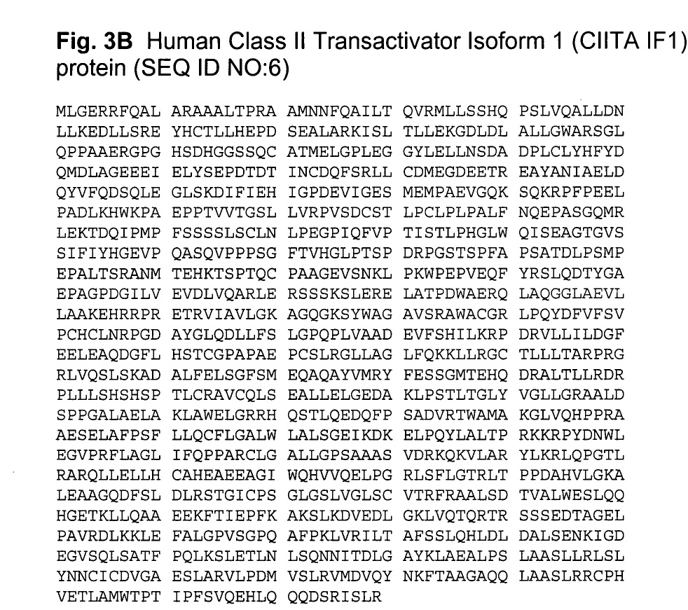

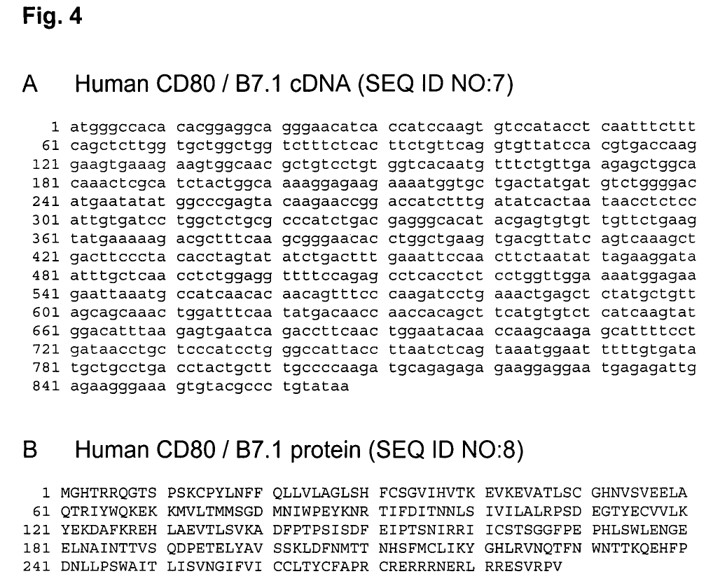

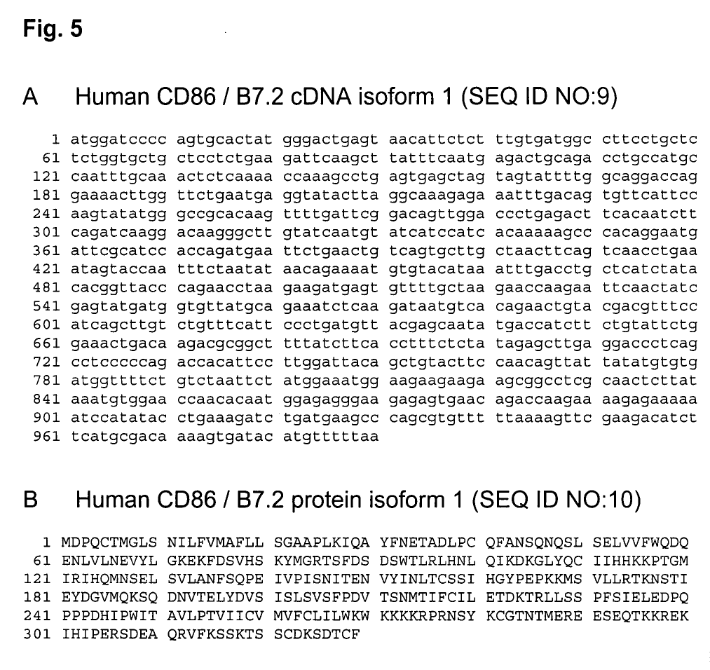

### DEFINITIONS

To facilitate an understanding of the present invention, a number of terms and phrases are defined below:

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as one or more of the desired activities or functional properties (e.g., enzymatic activity, ligand binding, signal transduction, etc.) of the full-length polypeptide are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

For instance, the term “CIITA gene” refers to the full-length CIITA nucleotide sequence. However, it is also intended that the term encompass fragments of the CIITA nucleotide sequence, as well as other domains (e.g., functional domains) within the full-length CIITA nucleotide sequence. Furthermore, the terms “CIITA gene,” “CIITA nucleotide sequence,” and “CIITA polynucleotide sequence” encompass DNA, cDNA, and RNA sequences.

As used herein, the terms “purified” and “isolated” refer to molecules (polynucleotides or polypeptides) that are removed or separated from their natural environment. “Substantially purified” molecules are at least 50% free, preferably at least 75% free, more preferably at least 90% and most preferably at least 95% free from other components with which they are naturally associated.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., βgal) or may provide a tool for isolation purposes (e.g., GST).

Suitable systems for production of recombinant proteins include but are not limited to prokaryotic (e.g., Escherichia coli), yeast (e.g., Saccaromyces cerevisiae), insect (e.g., baculovirus), mammalian (e.g., Chinese hamster ovary), plant (e.g., safflower), and cell-free systems (e.g., rabbit reticulocyte).

As used herein, the term “coding region” refers to the nucleotide sequences that encode the amino acid sequences found in the nascent polypeptide as a result of translation of an mRNA molecule. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

Where amino acid sequence is recited herein to refer to an amino acid sequence of a naturally occurring protein molecule, “amino acid sequence” and like terms, such as “polypeptide” or “protein,” are not meant to limit the amino acid sequence to the complete, native amino acid sequence associated with the recited protein molecule. The term “wild-type” refers to a gene or gene product that has the characteristics of that gene or gene product when isolated from a naturally occurring source. A wild type gene is that which is most frequently observed in a population and is thus arbitrarily designated the “normal” or “wild-type” form of the gene.

In contrast, the terms “modified,” “mutant,” and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

The ubiquitin family of proteins have a variety of functions including the non-specific ligation of polypeptides and protein-protein dimerization. The terms “ubiquitin,” “Ub,” “ubiquitin c,” “UBC” and “polyubiquitin,” as used herein refer to a human ubiquitin gene (e.g., Homo sapiens—GENBANK Accession No. NM_{—}021009) and its gene product, as well as its mammalian counterparts, including wild type and mutant products. In some preferred embodiments, the term “human Ub” refers to a single ubiquitin coding unit (e.g., SEQ ID NO:3), while the term “human Ub protein sequence” refers to an ubiquitin monomer (e.g., SEQ ID NO:4). Mammalian counterparts of the exemplary human Ub protein include but are not limited to monomers of nonhuman primate Ub proteins (e.g., Pan troglodytes—GENBANK Accession No. XP_{—}001136911.1), canine Ub proteins (e.g., Canis familiaris—GENBANK Accession No. XP_{—}853060.1), and rodent Ub proteins (e.g., Mus musculus—GENBANK Accession No. NP_{—}062613.2; and Rattus norvegicus—GENBANK Accession No. NP_{—}059010.1). Human Ub variants, which differ from the wild type human Ub sequences in preferably fewer than 10% of the residues, more preferably fewer than 5% of the residues and most preferably fewer than 1% of the residues may also be suitable for use in the methods and compositions of the present invention.

The class II transactivator is a positive regulator of class II major histocompatibility gene transcription. The terms “class II transactivator,” “C1ITA,” and “C2TA,” as used herein refer to a human CIITA gene (e.g., Homo sapiens—GENBANK Accession No. NM-000246) and its gene product, as well as its mammalian counterparts, including wild type and mutant products. The human CIITA isoform 1 (CIITA IF1) coding region is set forth as SEQ ID NO:5, while the human CIITA IF1 protein sequence is set forth as SEQ ID NO:6. Mammalian counterparts of the exemplary human CIITA protein include but are not limited to nonhuman primate CIITA proteins (e.g., Pan troglodytes—GENBANK Accession No. XP_{—}510813.2), canine CIITA proteins (e.g., Canis familiaris—GENBANK Accession No. XP_{—}547128.2), and rodent CIITA proteins (e.g., Mus musculus—GENBANK Accession No. NP_{—}031601.1; and Rattus norgegicus—GENBANK Accession No. NP_{—}445981.1). Human CIITA IF1 variants, which differ from the wild type human CIITA IF1 sequences in preferably fewer than 10% of the residues, more preferably fewer than 5% of the residues and most preferably fewer than 1% of the residues may also be suitable for use in the methods and compositions of the present invention.

The B-lymphocyte activation antigen B7.1 provides regulatory signals for T lymphocytes as a consequence of binding to the CD28 and CTLA4 ligands of T cells. The terms “CD80,” “B7.1” “CD28 antigen ligand 1,” and “B-lymphocyte activation antigen B7-1,” as used herein refer to a human B7.1 gene (e.g., Homo sapiens—GENBANK Accession No. NM_{—}005191) and its gene product, as well as its mammalian counterparts, including wild type and mutant products. The human B7.1 coding region is set forth as SEQ ID NO:7, while the human B7.1 protein sequence is set forth as SEQ ID NO:8. Mammalian counterparts of human B7.1 protein include but are not limited to nonhuman primate B7.1 proteins (e.g., Pan troglodytes—GENBANK Accession No. XP_{—}001163234.1), canine B7.1 proteins (e.g., Canis familiaris—GENBANK Accession No. XP_{—}001003147.1), and rodent B7.1 proteins (e.g., Mus musculus—GENBANK Accession No. NP_{—}033985.1; and Rattus norgegicus—GENBANK Accession No. NP_{—}037058.1). Human B7.1 variants, which differ from the wild type human B7.1 sequences in preferably fewer than 10% of the residues, more preferably fewer than 5% of the residues and most preferably fewer than 1% of the residues may also be suitable for use in the methods and compositions of the present invention.

The B-lymphocyte activation antigen B7.2 provides regulatory signals for T lymphocytes as a consequence of binding to the CD28 and CTLA4 ligands of T cells. The terms “CD86,” “B7.2” “CD28 antigen ligand 2,” and “B-lymphocyte activation antigen B7-2,” as used herein refer to a human B7.2 gene (e.g., Homo sapiens—GENBANK Accession No. NM_{—}175862 or GENBANK Accession No. NM_{—}006889) and its gene product, as well as its mammalian counterparts, including wild type and mutant products. The human B7.2 coding region is set forth as SEQ ID NO:9, while the human B7.2 protein sequence is set forth as SEQ ID NO:10. Mammalian counterparts of human B7.2 protein include but are not limited to nonhuman primate B7.2 proteins (e.g., Pan troglodytes—GENBANK Accession No. XP_{—}526283.2), canine B7.2 proteins (e.g., Canis familiaris—GENBANK Accession No. NP_{—}001003146.1), and rodent B7.2 proteins (e.g., Mus musculus—GENBANK Accession No. NP_{—}062261.2; and Rattus norgegicus—GENBANK Accession No. NP_{—}064466.1). Human B7.2 variants, which differ from the wild type human B7.2 sequences in preferably fewer than 10% of the residues, more preferably fewer than 5% of the residues and most preferably fewer than 1% of the residues may also be suitable for use in the methods and compositions of the present invention.

The terms “fragment” and “portion” when used in reference to a nucleotide sequence refers to partial segments of that sequence. The fragments may range in size from 10 nucleotides to the entire nucleotide sequence minus one nucleotide (e.g., at least 10, 25, 50, 100, 250, 500 or 1000 nucleotides, etc.).

Similarly, the terms “fragment” and “portion” when used in reference to a polypeptide sequence refers to partial segments of that sequence. In some embodiments, the portion has an amino-terminal and/or carboxy-terminal deletion as compared to the native protein, but where the remaining amino acid sequence is identical to the corresponding positions in the amino acid sequence deduced from a full-length cDNA sequence. The fragments may range in size from 4 amino acids long to the entire amino acid sequence minus one amino acid (e.g., at least 4, 10, 25, 50, 100, 250, 500 or 1000 amino acids, etc.).

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA. In contrast, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349, 293-299, 1991). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, a CD25-reactive antibody is an antibody, which binds to CD25.

As used herein, the term “immune response” refers to the reactivity of a subject's immune system in response to an antigen. In mammals, this may involve antibody production, induction of cell-mediated immunity, and/or complement activation. In preferred embodiments, the term immune response encompasses but is not limited to one or more of a “lymphocyte proliferative response,” a “cytokine response,” and an “antibody response.”

In particularly preferred embodiments, the immune response is largely reactive with tumor cells. For instance, when used in reference to administration of transfected tumor cells to a subject, the term refers to the immune response produced in the subject that reacts with the transfected tumor cells and in preferred embodiments with the parental untransfected tumor cells. Immune responses reactive with tumor cells are measured in vitro using various methods disclosed herein.

The term “reactive with an antigen of interest” when made in reference to an immune response refers to an increased level of the immune response to the antigen of interest (e.g., tumor cell) as compared to the level of the immune response to a control (e.g., irrelevant antigen).

The term “lymphocyte proliferative response” refers to transfected tumor cell-induced increase in lymphocyte numbers. Alternatively, or in addition, the term “proliferation” refers to the physiological and morphological progression of changes that cells undergo when dividing, for instance including DNA replication as measured by tritiated thymidine incorporation.

The term “cytokine response” refers to transfected tumor cell-induced cytokine secretion by lymphocytes as measured for instance by assaying culture supernatants for cytokine content (e.g., IL-2, IFNγ, TNFα, IL-4, etc) by ELISA.

The term “antibody response” refers to the production of antibodies (e.g., IgM, IgA, IgG) that bind to an antigen of interest (e.g., transfected or untransfected tumor cells), this response is measured for instance by assaying sera by antigen ELISA.

The term “adjuvant” as used herein refers to any compound that when injected together with an antigen, non-specifically enhances the immune response to that antigen. Exemplary adjuvants include but are not limited to incomplete Freunds adjuvant (IFA), aluminum-based adjuvants (e.g., AIOH, AIPO4, etc), and Montanide ISA 720.

The terms “diluent” and “diluting agent” as used herein refer to agents used to diminish the strength of an admixture. Exemplary diluents include water, physiological saline solution, human serum albumin, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

The terms “carrier” and “vehicle” as used herein refer to usually inactive accessory substances into which a pharmaceutical substance (e.g., transfected tumor cells) is suspended. Exemplary carriers include liquid carriers (such as water, saline, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins).

As used herein, the term “biologically active” refers to a molecule having structural, regulatory and or biochemical functions of a wild type molecule. For instance, a biologically active CIITA molecule is a homolog of a human CIITA molecule, while in other instance the biologically active molecule is a portion of a human CIITA molecule. Other biologically active molecules, which find use in the compositions and methods of the present invention include but are not limited to mutant (e.g., variants with at least one deletion, insertion or substitution) molecules. For instance CIITA biological activity is determined by restoration or introduction of CIITA activity in cells lacking CIITA activity, through transfection of the cells with a CIITA expression vector containing a CIITA gene, derivative thereof, or portion thereof.

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “nonconservative substitution” refers to a change in which an amino acid from one family is replaced with an amino acid from another family (e.g., replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.).

The terms “mammals” and “mammalian” refer to animals of the class mammalian that nourish their young by fluid secreted from mammary glands of the mother, including human beings. The class “mammalian” includes placental animals, marsupial animals, and monotrematal animals.

The terms “patient” and “subject” refer to a mammal that is a candidate for receiving medical treatment.

The term “control” refers to subjects or samples that provide a basis for comparison for experimental subjects or samples. For instance, the use of control subjects or samples permits determinations to be made regarding the efficacy of experimental procedures. In some embodiments, the term “control subject” refers to animals or cells receiving a mock treatment (e.g., empty vector).

### DETAILED DESCRIPTION OF THE INVENTION

Tumor immunotherapy is an alternative treatment option, particularly for cancer patients having a poor prognosis. In particular, the present invention provides improved methods and compositions for converting cancer cells into antigen presenting cells. The immunogenic compositions of the present invention are contemplated to be suitable for the treatment and prevention of solid tumors (e.g., non blood cell origin).

### I. Tumor Cell Evasion of Host Immune Response

A. Inadequate Costimulatory Signaling

A common defect in the recognition and eradication of tumor cells by T lymphocytes is the lack of appropriate antigen processing and presentation (APP) and/or the absence of suitable costimulatory signals from the tumor cells themselves. Indeed, tumor cells engineered to express costimulatory molecules can induce anti-tumor responses in several animal models of cancer (Chen et al., Cell, 7:1093-1102, 1992; Chen et al., J Exp Med, 179:523-532, 1994; Chen et al., Cancer Res, 54:5420-5423, 1994; Felzmann et al., Cancer Lett, 135:1-10, 1999; Hurwitz et al., Int J Cancer, 77:107-113, 1998; Martin-Fontecha et al., J Immunol, 164:698-704, 2000; Takahashi et al., Cancer Gen Ther, 7:144-150, 2000; Townsend and Alligson, Science; 259:368-370, 1993; Townsend et al., Cancer Res, 54:6477-6483, 1994; Vasilevko et al., Clin Exp Metastasis, 20:489-498, 2003; and Yang et al., J Immunol, 154:2794-2800, 1995).

B. Inadequate Antigen-Specific Signaling

In most cases however, the therapeutic efficacy of engineering tumor cells to express costimulatory molecules is limited, possibly due to lack of T helper cells, whose activation depends on the expression of major histocompatibility complex (MHC) class II molecules (Jang et al., Mol Cell, 13:130-136, 2002; and Pulaski and Ostrand-Rosenberg, Cancer Res, 58:1486-1493, 1998). Another limitation of this approach is the suboptimal APP by MHC class I on many tumor cells, such that the CD80- or CD86-transduced cells deliver a costimulatory signal to T cells in the absence of an antigen-specific signal. Although several attempts have been made to restore the expression of MHC determinants on tumor cells, the full restoration of APP has been more difficult since it requires additional accessory molecules. The identification of the class II transactivator (CIITA), which is the master coactivator for the transcription of genes required for APP, has been an important step forward in the art (LeibundGut-Landmann et al., Eur J Immunol, 34:1513-1525, 2004; and Ting et al., Cell, 109:S21-S33, 2002). CIITA not only induces the expression of the polymorphic structural α and β chains of MHC class II molecules but also induces expression of monomorphic DOA and DOB, as well as the DMA, DMB, and invariant chain (II) genes. In some cells, CIITA can also increase the expression of MHC class I genes (Girdlestone, Blood, 95:3804-3808, 2000; Liu et al., Hematol Oncol, 17:149-160, 1999; and Martin et al., Immunity, 6:591-600, 1997).

### II. Genetically Engineered Tumor Cells as Antigen Presenting Cells

While the present invention is not limited to any particular mechanism, and an understanding of the mechanism is not necessary to practice the present invention, engineering tumor cells to express CIITA is contemplated to reconstitute APP by both the MHC class I and class II compartments, and with the addition of costimulatory molecules is contemplated to transform these cells into potent antigen presenting cells (APCs). To improve upon prior art methods to convert tumor cells into APCs, transcriptional regulation of CIITA was examined in detail as described in Example 1.

A. Class II Transactivator (CIITA)

Since MHC class II determinants are involved directly in the establishment of adaptive immunity, it is not surprising that their expression is tightly regulated. This regulation takes place at the level of transcription and is mediated by the class II transactivator (CIITA). Transcription of CIITA can be initiated from three distinct promoters called PI, PII, and PIV, which direct the synthesis of three isoforms (IF) of CIITA: IF1, IF3 and IF4 (Muhlethaler-Mottet et al., EMBO J, 16:2851-2860, 1997). Different isoforms are expressed following distinct stimuli in different cells. Whereas CIITA IF1 is expressed in macrophages and myeloid dendritic cells (DCs), CIITA IF3 is expressed in B cells and plasmacytoid DCs (LeibundGut-Landmann et al., Nat Immunol, 5:899-908, 2004). Only one study has been performed on CIITA IF1 (Nickerson et al., J Biol Chem, 276:19089-19093, 2001). In contrast, CIITA IF3 has been studied in great detail (Greer et al., Nat Immunol, 4:1074-1082, 2003; Greet et al., J Immunol, 173:376-383, 2004; Li et al., Mol Cell Biol, 21:4626-4635, 2001; Schnappauf et al., Eur J Immunol, 33:2337-2347, 2003; Sisk et al., Int Immunol, 15:1195-1205, 2003; Spilianakis et al., Mol Cell Biol, 20:8489-8498, 2000; and Tosi et al., EMBO J, 21:5467-5476, 2002). CIITA isoforms do not bind to DNA. Rather they bind to the preformed enhanceosome on MHC class II promoters, where they recruit general transcription factors (Fontes et al., J Exp Med, 183:2517-2521, 1996; Fontes et al., Nucleic Acids Res, 25:2522-2528, 1997; Kanazawa et al., Immunity, 12:61-70, 2000; and Mahanta et al., Proc Natl Acad Sci USA, 94:6324-6329, 1997), as well as co-activators (Fontes et al., Mol Cell Biol, 19:941-947, 1999; Kretsovali et al., Mol Cell Biol, 18:6777-6783, 1998; Mudhasani and Fontes, Mol Cell Biol, 22:5019-5026, 2002; Spilianakis et al., supra, 2000; and Zika et al., Mol Cell Biol, 23:3091-3102, 2003) and function as transcriptional integrators.

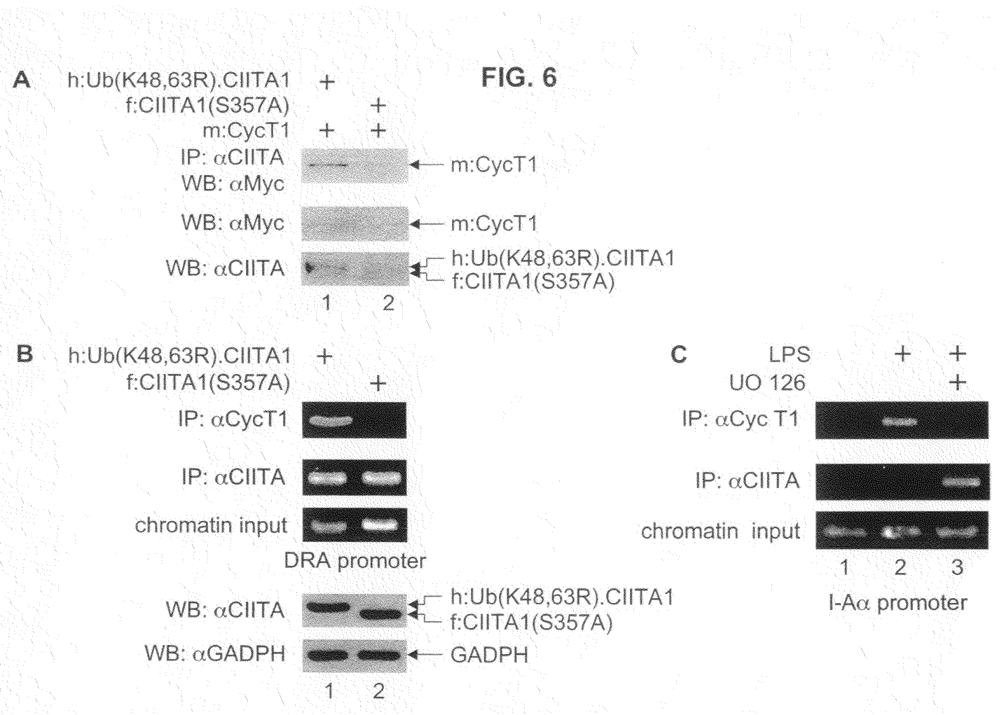

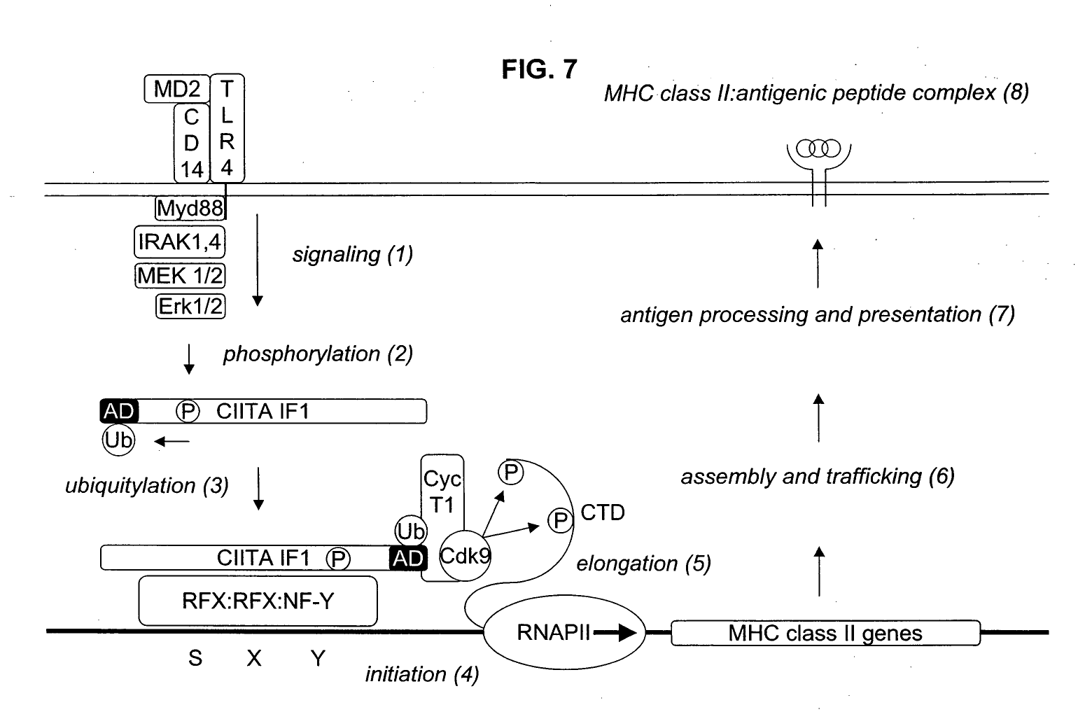

During development of the present invention, a mechanism that connects innate and adaptive immunity was identified. First, LPS was found to increase the expression of MHC class II genes in macrophages via the activation of Erk1/2, which was mediated by TLR4. Second, Erk1/2 phosphorylated CIITA IF1 on the serine at position 357. Third, this phosphorylation in a degron led to the monoubiquitylation of CIITA IF1. Fourth, by binding P-TEFb better, these modifications increased the transcriptional activity of CIITA IF1. Fifth, subsequent polyubiquitylation of CIITA IF1 resulted in its rapid degradation. These findings are summarized in the model presented in FIG. 6. Nonetheless, it is noted that knowledge of the mechanism(s) is not required in order to make and use the present invention. Indeed, the present invention is not limited to any particular mechanism.

Importantly, the finding that ubiquitylated CIITA IF1 (h:Ub.CIITA1) was about six-fold more active in transcription assays than its non-ubiquitylated counterpart f:CIITA1(S357A) indicates that h:Ub.CIITA1 is a superior candidate. In particular, heterologous expression of h:Ub.CIITA1 in tumor cells may be used, in some embodiments, to direct heightened expression of APP molecules (e.g., MHC class II molecules among others) in tumor cells as compared to the heterologous expression of h:CIITA1 in these cells.

B. CD80 (B7.1) and CD86 (B7.2) Costimulatory Antigens

In some embodiments, tumor cells are engineered to express a costimulatory molecule such as CD80 (Freeman et al., J Immunol, 143:2714, 1989; and Freeman et al., J Exp Med, 174:625, 1991) or CD86 (Freeman et al., Science, 262:909, 1993; and Azuma et al., Nature, 366:76, 1993) in addition to Ub.CIITA1. In this way the tumor-APCs of the present invention are suitable for providing signals to T cells via T cell receptors, as well as CD28 molecules. Providing both an antigen-induced first signal and a costimulatory signal to T lymphocytes enhances cell survival, cytokine production and differentiation into effector cells.

C. Cytokines

In further embodiments, subjects immunized with tumor-APCs (e.g., engineered to co-express Ub.CIITA1 and CD80 or CD86) are treated with adjunct immunomodulatory agent(s) such as cytokines or cytokine receptor-reactive antibodies to enhance T cell growth and activation. In other embodiments, tumor-APCs engineered to express Ub.CIITA1 and a costimulatory molecule are also transfected with an expression vector comprising a cytokine. In some embodiments, the cytokine is selected from the group consisting of interleukin-2 (IL-2), interferon-gamma (IFN-γ), and granulocyte macrophage-colony stimulating factor (GM-CSF). In further embodiments, the cytokine receptor-reactive antibody is reactive with the IL-2 receptor (CD25). In some embodiments the antibody is a monoclonal antibody (mAb), which in preferred embodiments is a human antibody, a humanized antibody or a chimeric antibody.

### III. Methods for Treating or Preventing Cancer

As described in Example 2, MCNeuA tumor cells are engineered to express high levels of MHC class II and CD80 molecules to create an anti-tumor composition (e.g., vaccine) against the development of mouse mammary tumors in MMTV-neu mice (Campbell et al., In Vitro Cell Dev Biol Anim, 38:326-333, 2002).

Expression of Ub.CIITA1 and CD80 or CD86 molecules in mouse mammary tumor cells is expected to reduce or abrogate their tumorigenicity. Furthermore, these cells when administered as a single injection to 6-week-old MMTV-neu transgenic mice are contemplated to confer significant protection from the subsequent development of spontaneous mammary tumors. In this way, mammary tumor cells can be genetically engineered as efficient APCs to immunize a subject against the development and growth of breast cancer. Although expression of Ub.CIITA1 alone may delay but not completely inhibit tumor growth (e.g., as compared to CD80′ or CD80 plus Ub.CIITA1). This could be due to the need for the co-expression of a costimulatory molecule such as CD80 or due to a loss of MHC class II expression over time in vivo. Repeated immunizations (with cells expressing Ub.CIITA1 and CD80) are contemplated to circumvent this potential problem.

In vivo animal immunotherapy models using tumor cells transfected with CIITA have v been reported for a mouse sarcoma, SaI (Armstrong et al., Proc Natl Acad Sci USA, 94:6886-6891, 1997), lung carcinoma, Line 1 (Martin et al., J Immunol, 162:6663-6670, 1999), and mammary adenocarcinoma, TS/A (Meazza et al., Eur J Immunol, 33:1183-1192, 2003). Transfection of CIITA resulted in the expression of MHC class II molecules in each of these models. However, effects of CIITA expression on tumor growth in vivo varied. For example, SaI tumor cells expressing CIITA or only structural MHC class II determinants were not or were rejected when injected into mice, respectively. In contrast to these two studies, the expression of CIITA in TS/A mammary carcinoma cells significantly reduced their tumorigenicity and mice that rejected TS/A-CIITA cells were resistant to a subsequent challenge with parental TS/A tumor cells. The differences in results between these four studies may be due to differences in the types of tumors used, variable levels of expression of the transduced genes and/or the extinction of MHC class II on tumor cells.

However, when taken together with the observations made during development of the present invention, these finding indicate that converting tumor cells into APCs, tumor-APCs, is a useful immunotherapeutic strategy. In tumor-APCs, the presentation of endogenous tumor antigens is expected to improve, especially since the APP machinery is turned on maximally even when exogenous antigens, such as bacteria, are not encountered. Some of these endogenous antigens might also enter tumor-APCs by endocytosis or pinocytosis of exosomes and debris from apoptotic cells. Although live cells are used to provide continuous antigenic stimulation in the studies described in Example 2, the present invention is not limited to the use of live cells. In fact, in other embodiments, inactivated tumor-APCs, transduced ex vivo with Ub.CIITA1, costimulatory molecules (CD80 or CD86), and/or cytokines or chemokines, are utilized in preclinical and clinical situations. Alternatively, viral or non-viral systems could be used to deliver Ub.CIITA1 and CD80 expression constructs directly to tumor cells in vivo. Since the expression of these genes should lead to an immune response against the tumor, the delivery system would not have to be 100% efficient. Moreover, transduction of only a fraction of the tumor may be sufficient to elicit an immune attack against the untransfected cells.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); rmol (nanomoles); g (grams); mg (milligrams); μg (micrograms); ng (nanograms); I or L (liters); ml (milliliters); μl (microliters); cm (centimeters); mm (millimeters); m (micrometers); nm (nanometers); ° C. (degrees Centigrade); U (units), mU (milliunits); min. (minutes); sec. (seconds); % (percent); kb (kilobase); bp (base pair); PCR (polymerase chain reaction); MHC (major histocompatibility complex); APC (antigen presenting cells); CIITA IF1 or CIITA1 (class II transactivator isoform 1); and Ub (ubiquitin).

### Example 1

### Modification in CIITA Isoform 1 Stimulate MHC Class II Transcription

The following experiments were performed to analyze the signaling cascade from TLR4 to CIITA isoform 1, which connects innate and adaptive immunity in macrophages. The results obtained were then employed in the design of methods and compositions for engineering tumor cells to express high levels of MHC class I and II molecules.

### Animals, Cells and Cell Culture

C57BL/10ScN mice were purchased from the Jackson Laboratory (Bar Harbor, Me.) and bred in a colony at UCSF (San Francisco, Calif.). HeLa, COS and RAW 264.7 cells were maintained in standard conditions. Bone marrow-derived macrophages (BMDMs) were prepared and maintained as described previously (Nishiya and DeFranco, J Biol Chem, 279:19008-19017, 2004). LPS Re 595 was obtained from Sigma (St. Louis, Mo.). MEK1/2 inhibitor UO126 was obtained from Promega (Madison, Wis.) and proteasome inhibitor ALLN was purchased from Calbiochem (La Jolla, Calif.). λ-phosphatase was obtained from Sigma (St. Louis, Mo.).

### Plasmid DNAs

Reporter plasmid pDRASCAT was described previously (Tosi et al., EMBO J, 21:5467-5476, 2002). Plasmid m:Ub was described previously (Kurosu and Peterlin, Curr Biol, 14:1112-1116, 2.004). To construct a plasmid m:CycT1, the coding sequence of CycT1 was cloned into the expression vector pEF-myc between EcoRI and XbaI restriction sites. Plasmids f:CIITA1 and h:CIITA1 were gift from Dr. Ting (University of North Carolina, Chapel Hill, N.C., USA) and Dr. Chang (Indiana University School of Medicine, Indianapolis, Ind., USA). To construct a plasmid coding for f:CIITA1(S357A), the f:CIITA1 plasmid was subjected to site-directed mutagenesis with the QuickChange II XL Site-Directed Mutagenesis Kit (Stratagene, La Jolla, Calif.) according to the manufacturer's instructions. To construct plasmids coding for the h:Ub.CIITA1 and h:Ub(K48,63R). CIITA1, the Ub and Ub(K48,63R) plasmids were digested with EcoRI and cloned into h:CIITA1 (HA tagged CIITA IF1 in the pcDNA3 plasmid). Murine TLR4 was cloned into the pMX-pie bicistronic retroviral vector, as described previously (Onishi et al., Exp Hematol, 24:324-329, 1996). All plasmids were verified by DNA sequencing.

### Immunoreagents

The monoclonal anti-CIITA (sc-13556), anti-Ub (sc-8017), and anti-Myc (9E10) antibodies, and the polyclonal anti-Cyclin T1 (sc-8127), anti CIITA (sc-9870 and sc-9869), and anti-Erk1/2 antibodies were obtained from Santa Cruz Biotechnology (Santa Cruz, Calif.). The monoclonal anti-phospho Erk1/2 antibody was purchased from Cell Signaling Technology (Beverly, Mass.). The monoclonal anti-1-Aα (KH 118) antibody was obtained from Becton Dickinson (San Diego, Calif.). The monoclonal anti-FLAG M2 antibody and the anti-FLAG M2 beads were purchased from Sigma.

### Viral Production and Infection

Retroviruses were produced by triple transfection of HEK293T cells with retroviral constructs along with gag-pol and vesicular stomatitis virus G glycoprotein expression constructs (Yee et al., Proc Natl Acad Sci USA, 91:9564-9568, 1994). BMDMs were infected as described previously (Nishiya and DeFranco, supra, 2004).

### Transient Transfection and CAT Assay

Cells were seeded into 100-mm-diameter petri dishes approximately 12 hr prior to transfection. Cells were transfected with FuGene6 reagent according to the manufacturer's instructions (Gibco-BRL, Rockville, Md.). Chloramphenicol acetyltransferase (CAT) enzymatic assays were performed as described (Fujinaga et al., J Virol, 72:7154-7159, 1998). Fold trans-activation represents the ratio between the CIITA-activated transcription and the activity of the reporter plasmid alone.

### Immunoprecipitation Assay and Western Blot Analysis

Cells were transfected with 2 μg of indicated plasmid vectors. About 18 hours after transfection, immunoprecipitations were performed as described previously (Tosi, supra 2002). Precipitated proteins were resolved on SDS-PAGE and analyzed by immuno-blotting with the appropriate antibody followed by HRP-conjugated secondary antibody. Blots were developed by, chemiluminiscence assay from Perkin Elmer Life Sciences (Boston, Mass.).

### Chromatin Immunoprecipitation (ChIP) Assays, RT-PCR and QPCR

ChIP assays were performed as described previously (Jiang et al., Mol Cell Biol, 25:10675-10683, 2005). RNA was extracted from RAW 264.7 cells using the Trizol protocol from Invitrogen (Carlsbad, Calif.). RT-PCR was described previously (Lin et al., J Virol, 77:8227-8236, 2003). cDNAs were then amplified by primers described previously (Takeuchi et al., J Am Soc Nephrol, 14:2823-2832, 2003). Quantitative PCR was performed using a Stratagene Mx3005P QPCR System, according to the manufacturer's protocol.

### In Vitro Transcription and Translation and In Vitro Kinase Assay

f:CIITA1 and f:CIITA1(S357A) were expressed in vitro by using coupled rabbit reticulocyte lysate transcription and translation system from Promega (Madison, Wis.). In vitro kinase assay with Erk1/2 was performed according to the manufacturer's instructions (Upstate biotechnology Lake Placid, N.Y.).

### Pulse-Chase Analysis

Approximately 18 hours after transfection, pulse-chase analysis was performed as described previously (Greer et al., J Immunol, 173:376-383, 2004). Precipitated proteins were resolved on SDS-PAGE and analyzed by radiography.

### LPS Increases the Expression of MHC Class II Determinants Via TLR4 in Mouse Macrophages

Upon stimulation with LPS, macrophages increase the surface levels of MHC class II determinants. Via TLR4, LPS also activates Erk1/2, JNK and p38MAP kinases. As described herein, the influence of these kinases and TLR4 on expression of MHC class II determinants was assessed. The mouse macrophage cell line RAW 264.7 was stimulated with LPS for two hours or with solvent as a negative control. After twenty-four hours, the levels of MHC class II determinants were measured. To exclude possible trans-location of MHC class II determinants from cytoplasm to the cell surface, their levels were determined by western blotting rather than by FACS. LPS clearly induced the expression of MHC class II determinants, and this activation was correlated with the activation of Erk1/2. In contrast, when cells were treated with the MEK inhibitor UO 126 prior to the addition of LPS, the expression of MHC class II determinants was comparable to that in un-stimulated cells. UO 126 is a specific MEK1/2 inhibitor, which prevents the phosphorylation and subsequent activation of Erk1/2. The same experiments were performed with inhibitors of JNK and p38MAP kinases, but they had no effect on the induction of MHC class II determinants by LPS. Thus, LPS was found to induce the expression of MHC class II determinants via Erk1/2.

To determine the effects of LPS on levels of MHC class II mRNA, RAW 264.7 cells were stimulated with LPS prior to analysis of I-Aalpha expression by RT-PCR (at time points 0, 0.5, 1, 2, 4 and 8 hrs). There was no MHC class II mRNA detected in un-stimulated cells. MHC class II mRNA appeared at 1 hour and peaked 2 hours after the addition of LPS. Four hours later, MHC class II transcripts disappeared almost completely, while the amounts of actin mRNA used as the internal control were equivalent in all samples. Thus, LPS induces the transcription of MHC class II genes.

Different isoforms of CIITA have been found in RAW 264.7 cells (Giroux et al., J Immunol, 171:4187-4194, 2003; and Nikcevich et al., J Neuroimmunol, 99:195-204, 1999). The cytoplasm of RAW 264.7 cells was analyzed for the presence of CIITA IF1, IF3 and IF4 transcripts by RT-PCR. In un-stimulated cells, low levels of CIITA IF1 and high levels of putative CIITA IF4 mRNA were detected. Stimulation with LPS for two hours resulted in the complete disappearance of CIITA IF1, and a slight increase of putative CIITA IF4 mRNA levels. However, CIITA IF3 mRNA and MHC class II transcripts were not detected, indicating that the putative CIITA IF4 mRNA was not translated in resting cells. Moreover, the promoter region of CIITA IF4 was not transcribed. Finally, levels of RT PCR products from intron 1 correlated with those of the putative CIITA IF4. Importantly, the control PCR without RT excluded the possibility of genomic contamination of samples. Thus the primers used herein amplified sterile CIITA rather than coding CIITA IF4 transcripts. In support of this notion, CIITA IF1 was the only isoform detected by immunoprecipitation and subsequent western blotting in RAW 264.7 cells. Similar aberrant transcription and/or splicing of CIITA transcripts had been reported in B cell lines (Riley et al., Immunity, 2:533-543, 1995). Thus, only CIITA IF1 transcripts mediate functional CIITA protein in RAW 264.7 cells. Next, quantitative real time RT-PCR was performed to quantify mRNA levels of CIITA IF1 in stimulated and un-stimulated cells. The stimulation with LPS for two hours decreased levels of CIITA IF1 mRNA up to 7-fold, compared to its levels in un-stimulated cells. Thus, LPS induces the transcription of MHC class II genes without a concomitant increase in CIITA IF1 transcripts.

To determine if TLR4 was required for these effects, primary bone marrow derived macrophages from TLR4^{-/-} mice were infected with an adenovirus expressing TLR4 or an empty adenoviral vector as the control. Two days later, macrophages were stimulated with LPS or left un-treated. Indeed, LPS induced the expression of MHC class II determinants only on macrophages that expressed TLR4, which correlated with the activation of Erk1/2 (e.g., phosphorylation). The overall levels of Erk1/2 were comparable in all samples. Thus LPS increases the expression of MHC class II determinants in macrophages via the activation of Erk1/2. Moreover, this stimulation depends on TLR4, is not due to increased synthesis of CIITA IF1, and results in a transient induction of transcription of MHC II genes. Erk1/2 phosphorylates CIITA IF1 on serine 357 Effects of LPS on levels of MHC class II determinants depended on the activation of Erk1/2. Moreover, CIITA IF1 directs the transcription of MHC class II genes in macrophages. However, since amounts of CIITA IF1 mRNA actually decreased, Erk1/2 was contemplated to modify the preformed CIITA IF1 for its increased transcriptional activity. When analyzed by gel electrophoresis, CIITA IF3 migrates as a double band, which has been attributed to its phosphorylation (Greer et al., J Immunol, 173:376-383, 2004; Sisk et al., Int Immunol, 15:1195-1205, 2003; and Tosi et al., EMBO J, 21:5467-5476, 2002). Moreover, treatment with λ phosphatase and direct labeling revealed that the upper band represents the phosphorylated form of CIITA IF3 (Tosi et al., supra, 2002). Since it also appears as a double band, CIITA IF1 was analyzed to determine whether it too is modified by phosphorylation. The FLAG epitope-tagged CIITA IF1 protein (f:CIITA1) was expressed in COS cells. An aliquot of the cell lysate was incubated with λ phosphatase, which removes phosphates from serines and threonines. As contemplated, the upper band in f:CIITA1 disappeared completely. Thus, CIITA IF1 is also phosphorylated in cells.

Since the increased expression of MHC class II determinants after LPS stimulation was found to depend on the activation of Erk1/2, putative Erk1/2 phosphorylation sites in CIITA IF1 were identified. Erk1/2 is a proline directed kinase with the consensus phosphorylation sequence PX(S/T)P, where the serine or threonine is phosphorylated. Indeed, there is only one consensus Erk1/2 phosphorylation site in CIITA IF1, which contains a serine at position 357 (S357). To determine whether S357 is phosphorylated, a mutant f:CIITA1 protein in which the serine at position 357 is changed to alanine f:CIITA1(S357A) and can no longer be phosphorylated was produced. Both the wild type f:CIITA1 and the mutant f:CIITA1(S357A) proteins were expressed in COS cells. Interestingly, expression levels of the mutant f:CIITA1(S357A) protein were four-fold higher than those of the f:CIITA1 protein. Moreover, the mutant f:CIITA1(S357A) protein migrated as a single lower band, representing the non-phosphorylated form of CIITA IF1. Thus, CIITA IF1 is phosphorylated on S357 in cells.

In order to determine if Erk1/2 is involved in the phosphorylation of CIITA IF1, f:CIITA1 was expressed in COS cells treated with UO 126 or the solvent as a control. In the presence of UO 126 the upper band of f:CIITA1 disappeared, which correlated with the absence of phosphorylated Erk1/2. This finding indicates that Erk1/2 is involved in the phosphorylation of CIITA IF1.

Next, in vitro kinase assays were performed with Erk1/2. f:CIITA1 and the mutant f:CIITA1(S357A) proteins were transcribed and translated using rabbit reticulocyte lysate in vitro. Aliquots of the reaction were incubated with Erk1/2 or with the reaction buffer as the control. Indeed, as indicated by the appearance of the upper band, Erk1/2 phosphorylated f:CIITA1 protein. In contrast, Erk1/2 did not phosphorylate the mutant f:CIITA1(S357A) protein, which was confirmed by the absence of the upper band. Thus, Erk1/2 phosphorylates S357 in CIITA IF1 in vitro and in vivo.

### CIITA IF1 is Ubiquitylated in Cells

As described above, Erk1/2 phosphorylates S357 in f:CIITA1, and phosphorylation affects the stability of f:CIITA1. Namely, the expression levels of the mutant f:CIITA1(S357A) protein were higher than those of f:CIITA1. Phosphorylation and degradation of CIITA IF1 was contemplated to involve ubiquitin. f:CIITA1 and the mutant f:CIITA1(S357A) proteins were expressed on COS cells and subjected to pulse-chase analysis. Indeed, the mutant f:CIITA1(S357A) protein had a half-life of approximately two hours, which was 4 times longer than that of f:CIITA1. Thus phosphorylation of CIITA IF1 leads to its degradation.

Next, f:CIITA1 and the mutant f:CIITA1(S357A) proteins were expressed in COS cells. Transfected cells were treated with the proteasomal inhibitor ALLN or solvent as a control. ALLN increased levels of the phosphorylated form of f:CIITA1 approximately four-fold. It had almost no effect on levels of the mutant f:CIITA1(S357A) protein. Hence, phosphorylated CIITA IF1 is degraded via the proteasome.

To determine if CIITA IF1 is ubiquitylated, ubiquitylation assays were performed in vivo. In COS cells, f:CIITA1 was co-expressed with the Myc epitope-tagged ubiquitin (m:Ub) or an empty plasmid vector as a control. In the presence of f:CIITA1 and m:Ub, there was a strong ubiquitylation ladder. Since the mutant f:CIITA1(S357A) protein was more stable than f:CIITA1, the ubiquitylation of f:CIITA1 was contemplated to be dependent on the phosphorylation of S357. Ubiquitylation assays were also performed in vivo with the mutant f:CIITA1(S357A) protein. By densitometry the mutant f:CIITA1(S357A) protein was ubiquitylated 4-fold less than f:CIITA1. Thus CIITA is ubiquitylated and this modification is promoted by the phosphorylation of S357 in CIITA.

Ubiquitylation assays were repeated in RAW 264.7. Because of low levels of expression or accelerated degradation, CIITA IF1 or its ubiquitylation was not detected in un-treated cells, or cells treated with LPS and UO 126, respectively (data not presented). However, the heavily ubiquitylated CIITA IF1 was detected only when cells were pretreated with ALLN for 2 hours prior to the treatment with LPS for 40 minutes. Thereafter, cells were treated with ALLN for an additional 2 hours. Moreover, it was present in both phosphorylated and un-phosphorylated forms. Thus, endogenous CIITA IF1 in RAW 264.7 cells is phosphorylated and ubiquitylated.

In CIITA IF1, there is a predicted PEST sequence from positions 355 to 385 (PTSPDRPGSTSPFAPSATDLPSMPEPALTSR, set forth as SEQ ID NO: 11). The phosphorylation of these sequences decreases the stability of proteins (Muratani and Tansey, Nat Rev Mol Cell Biol, 4:192-201, 2003; and Yeh et al., Nat Cell Biol, 6:308-318, 2004). Since the phosphorylation of S357, which is flanked by a proline, serine and an acidic residue, led to the degradation of CIITA IF1, the actual PEST sequence in CIITA IF1 is extended on its N-terminus. Thus, S357 becomes a part of the real degron, which is located from positions 355 to 385 in CIITA IF1. In short, phosphorylation of S357, which is a part of a degron, precedes the ubiquitylation of CIITA IF1 that subsequently leads to its degradation.

Ubiquitylated CIITA IF1 Protein is more Active than its Counterpart that is not Ubiquitylated

Two post-translational modifications in CIITA IF1, namely phosphorylation and ubiquitylation, were found to be associated with the stability of CIITA IF1. It is known that ubiquitylation strongly influences the function of activators (Kurosu and Peterlin, Curr Biol, 14:1112-1116, 2004; Salghetti et al., EMBO J, 18:717-726, 1999; Salghetti et al., Proc Natl Acad Sci USA, 97:3118-3123, 2000; Salghetti et al., Science, 293:1651-1653, 2001; and Yeh, Nat Cell Biol, 6:308-318, 2004). Of note monoubiquitylation increases the activity of transcription factors, whereas polyubiquitylation leads to their degradation. Thus, monoubiquitylated CIITA IF1 protein is contemplated to be more active than its non-ubiquitylated counterpart.

It had been demonstrated previously that transcription factors fused to ubiquitin behave as endogenously ubiquitylated proteins (Kurosu and Peterlin, supra, 2004; and Salghetti et al., supra, 2001). With this in mind, HA epitope-tagged fusion protein between CIITA IF1 and ubiquitin (h:Ub.CIITA1) were constructed. Both f:CIITA1 and the h:Ub.CIITA1 fusion proteins were expressed in COS cells (FIG. 6A). Of interest, the steady state levels of h:Ub.CIITA1 chimera were ten-fold lower than those of f:CIITA1. This indicates that the h:Ub.CIITA1 fusion protein was extremely unstable. Indeed, pulse-chase analyses revealed that the half-life of the h:Ub.CIITA1 chimera was less than 10 minutes (FIG. 6B). As expected, the proteasomal inhibitor ALLN increased levels of the h:Ub.CIITA1 chimera substantially (FIG. 6C). Thus, the h:Ub.CIITA1 chimera is apparently rapidly degraded via the proteasome.

To compare the activities of the non-ubiquitylated and ubiquitylated CIITA IF1 proteins, transcriptional assays were done in transfected cells. The mutant f:CIITA1(S357A) protein represented the non-ubiquitylated and the h:Ub.CIITA1 fusion protein the ubiquitylated CIITA IF1 proteins, respectively. These proteins were co-expressed with pDRASCAT reporter plasmid in COS cells. Prior to performing CAT assays, the levels of both proteins were equalized. These results indicated that the ubiquitylated CIITA IF1 was approximately six-fold more active than its non-ubiquitylated counterpart (FIG. 6D), and hence that the ubiquitylation of CIITA IF1 increases its transcriptional activity.

Next the mutant Ub(K48,63R) protein, where lysines at positions 48 and 63 in ubiquitin were changed to arginines, which do not support polyubiquitylation, was linked to CIITA IF1 (mutant h:Ub(K48,63R). CIITA1 chimera). The Myc epitope-tagged CycT1 protein (m:CycT1) was co-expressed with the mutant h:Ub(K48,63R).CIITA1 chimera and the mutant f:CIITA1(S357A) protein in COS cells. CIITA1 proteins were immunoprecipitated with the anti-CIITA antibody, the membrane blotted with the anti-Myc antibody. Whereas the h:Ub(K48,63R).CIITA1 fusion protein clearly bound m:CycT1, the mutant f:CIITA1(S357A) did not. The inputs of m:CycT1 and CIITA1 proteins were comparable. Thus monoubiquitylation of CIITA IF1 precedes its binding to CycT1.

To determine if monoubiquitylated CIITA IF1 recruits CycT1 to MHC class II promoters, chromatin immunoprecipitation assays were performed. The mutant h:Ub(K48,63R). CIITA1 chimera and the mutant f:CIITA1(S357A) protein were expressed in HeLa cells. After cross-linking and sonication, immunoprecipitations with the anti-CycT1 and anti-CIITA antibodies were done. Immunoprecipitated DNA was amplified by PCR with specific primers complementary to the DRA promoter. When immunoprecipitated with the anti-CycT1 antibody, PCR products were present in cells expressing the mutant h:Ub(K48,63R).CIITA1 chimera but not in cells expressing the mutant f:CIITA1(S357A) protein. However, when immunoprecipitated with the anti-CIITA antibody, PCR products were comparable in cells expressing the mutant h:Ub(K48,63R).CIITA1 chimera and the mutant f:CIITA1(S357A) protein. The monoubiquitylated CIITA IF1 protein was contemplated to recruit P-TEFb to MHC class II promoters in cells.

To further extend these results, chromatin immunoprecipitation assays were done in RAW 264.7 cells with the endogenous CIITA IF1 protein and CycT1. RAW 264.7 cells were stimulated with LPS, LPS and U0126, or left untreated. After cross-linking and sonication, immunoprecipitations were performed with anti-CIITA and anti-CycT1 antibodies. Immunoprecipitated DNA was amplified by PCR with primers complementary to the I-Aα promoter. When immunoprecipitated with anti-CycT1 antibodies, PCR products were present only in cells, which were stimulated with LPS. In contrast, when immunoprecipitated with anti-CIITA antibodies PCR products were detected only in cells treated with UO126. Treatment with UO126 led to the stabilization and subsequent accumulation of CIITA IF1 on MHC class II promoters. However, it prevented its activation and binding to CycT1. In conclusion, the monoubiquitylated CIITA IF1 protein recruits P-TEFb to and subsequently enables the transcription from the MHC class II promoters in cells. Nonetheless, knowledge of the mechanism is not required in order to make and use the present invention.

### Example 2

### Tumor Cell Immunization of Mice

The following experiments are performed to convert tumor cells into antigen presenting cells expressing high levels of MHC II and CD80 molecules for use in the methods and compositions of the present invention for inducing a cellular immune response against tumor cells in a rodent model of breast cancer. In some embodiments, the methods and compositions of the present invention are suitable for use in treating and/or preventing cancer.

### Retroviral Constructions

cDNAs encoding the N-terminal Flag epitope-tagged human CIITA (hCIITA) and mouse B7.1 (mCD80) proteins are subcloned into the EcoRI site of the pWZLblast2 bicistronic retrovirus, which also encodes resistance to the antibiotic blasticidin. Likewise, cDNAs encoding h:Ub.CIITA1 and B7.1 proteins are subcloned into the bicistronic retrovirus. Recombinant amphotropic retroviruses are generated by transient transfection of Phoenix A packaging cells (PA317), as described previously (Morgenstern and Land, Nucleic Acids Res, 18:3587-3596, 1990; and Pear et al., Proc Natl Acad Sci USA, 90:8392-8396, 1993).

### Transgenic Mice and Mammary Carcinoma Cell Line

The FVB/N-TgN (MMTV-neu) N202 (denoted MMTV-neu) transgenic mice carry the wild-type rat neu transgene under the control of the mouse mammary tumor virus (MMTV) long terminal repeat (LTR) (Guy et al., Proc Natl Acad Sci USA, 89:10578-10582, 1992). Female MMTV-neu mice develop mammary lesions that are similar to human breast cancer. The MCNeuA mammary carcinoma cell line was established from a tumor that arose in a female MMTV-neu mouse (Campbell et al., In Vitro Cell Dev Biol Chem, 38:326-333, 2002). This cell line forms tumors when transplanted into syngeneic MMTV-neu mice. Cell culture and retroviral infection of cells MCNeuA and PA317 cell lines are maintained in DMEM supplemented with 10% (v/v) heat-inactivated fetal calf serum (FCS), 100 mM L-glutamine and 50 μg each penicillin and streptomycin per ml. All cells are grown in an atmosphere of 5% CO₂ at 37° C. PA317 cells are plated 12 hrs prior to transfection in 100 mm tissue culture dishes. Monolayers of MCNeuA cells are transfected with 10 μg of pWZLblast2 (M cells), pWZLblast2:hCIITA (MC cells), pWZLblast2:mCD80 (MB cells), or pWZLblast2:h:Ub.CIITA1(MU cells) retroviral plasmid vectors in OptiMEM media for 5 hrs using Lipofectamine (Life Technologies, Grand Island, N.Y.). Media is changed 24 hrs post-transfection (no antibiotics), and cells are allowed to grow for an additional 48 hrs. Twenty-four and 48 hrs supernatants are collected and cellular debris is removed by filtration. Monolayers of MCNeuA cells in mid-log growth phase are infected by the addition of 50% fresh viral supernatant and 8 μg/ml polybrene (Sigma, St. Louis, Mo.) for 12 hrs. The infection is repeated with supernatants collected at 48 hrs. Media are changed and cells are allowed to grow for an additional 48 hrs before selection in 24-well dishes. Cells are selected in DMEM with 10% FCS containing blasticidin (25 μg/ml). A pooled population of cells expressing CIITA or CD80 was trypsinized, expanded and subsequently cloned by limiting dilution. To express both CIITA and CD80 (MBC cells), CIITA selected clones cells are subsequently infected with the CD80 retrovirus. Likewise to express both h:Ub.CIITA1 and CD80 (MBU cells), h:Ub.CIITA1 selected clones cells are subsequently infected with the CD80 retrovirus. Double expressors are sorted by flow cytometry and single clones isolated by limiting dilution.

Growing tumors are removed from the MMTV-neu mice, washed in PBS at 4° C., cut into small pieces (2-4 mm), and incubated with of mixture of trypsin (0.25%) and collagenase in DMEM serum-free media at 37° C. for 3 hrs. The reaction is stopped by the addition of 10% fetal calf serum and separated cells are filtered through a fine mesh cloth. The cell suspension is then washed in PBS, plated in the complete DMEM medium with serum and allowed to grow for 24 hrs. Cells are analyzed as above.

### Protein Extracts, Immunoprecipitations and Western Blotting

Cells are lysed in lysis buffer containing 20 mM Tris, 137 mM NaCl, 5 mM EDTA, 0.5% (w/v) NP-40, and a protease inhibitor cocktail (Jabrane-Ferrat et-al., Mol Cell Biol, 22:5616-5625, 2002). The amount of protein is quantified with the BCA assay kit (Pierce, Location). Equivalent amounts of proteins are precleared with protein A-conjugated Sepharose beads, and subjected to immunoprecipitation using an anti-Flag M2 monoclonal antibody (mAb) (Sigma). Immunoprecipitated complexes are resolved by sodium dodecyl sulfate-polyacrylamide gels (7.5% PAGE) and analyzed by Western blotting with the anti-Flag M2 mAb. After washing under stringent conditions, immune complexes are detected using the horseradish peroxidase-conjugated anti-mouse IgG antibody (Amersham) and Enhanced chemiluminescence ECL-Plus, detection kit (NEN Life Science Products, Boston, Mass.).

### Flow Cytometry and Immunohistochemistry

The mAbs used for these studies are: anti-CD80 (PharMingen, San Diego, Calif.), anti-MHC class II (anti-IEβ/IAβ, which reacts with d, b, p, q, u, and j haplotypes) (provided by Dr. R. Accolla, Verona, Italy), and anti-c-Neu (Ab-4, Oncogene Sciences). Secondary Abs used are goat anti-mouse IgG-conjugated to either FITC or PE. Single-cell suspensions or monolayer cells grown on tissue culture chamber slides are washed in PBS containing 1% mouse serum, stained with primary and secondary Ab, and then analyzed. Flow cytometry is performed on a FACS calibur (Becton Dickinson Immunocytometry Systems, San Jose, Calif.). Dead cells are excluded by their uptake and staining with propidium iodide. Cells attached to tissue culture slides are analyzed by fluorescence microscopy (Nikon, Japan).

### Tumorigenicity of Cell Lines

Cells at 50-80% confluency are harvested by trypsinization, washed, and resuspended at 2.5×10⁷ cells/ml in PBS. Two hundred microliters (5×10⁶ cells) are injected SC into the hind leg of 6-8 weeks old female MMTV-neu mice. Mice are then monitored for tumor growth every other day starting at one week post-injection. Tumor measurements (length and width) are recorded using a caliper. Tumor volume is calculated as (length×width2)/2.

### Lymphocyte Purification and Growth

Single-cell suspensions are prepared from harvested spleens of 8-10 week-old MMTVneu female mice using a 40 μM nylon cell strainer (Becton Dickinson). After lysis of red blood cells with ACK lysing solution (Biofluids, Rockville, Md.), splenocytes in PBS are washed in PBS, layered onto Ficoll-Paque solution (Pharmacia) and are centrifuged for 20 min at 2,000 rpm at 4° C. Live cells are washed and non-adherent mononuclear leukocytes are collected after depletion of adherent cells by incubation for 3 hrs at 37° C. in tissue culture flasks. The enrichment of each subset of T cells is assessed by flow cytometry (Jabrane-Ferrat et al., FASEB J, 13:347-353, 1999). MCNeuA-derived cells are plated to 80-90% confluency 24 hrs prior to the assay in 96-well flat-bottom plates (Costar, Gaithersburg, Md.), and are irradiated with 3000 Rads 3-4 hrs prior to use as APC. Purified lymphocytes are treated for 3 hrs with 0.5 mM PMA plus 0.1 mM lonomycin and are then plated at 5×10⁴ cells/well onto a monolayer of irradiated mammary cells in a final volume of 200 μl/well. Media are changed every 2-3 days for a total of 6 days. Cultures are pulsed with 1 μCi of [³H]-thymidine and are harvested 16 hrs later onto glass filters using a 96-well plate harvester. Incorporated radioactivity is measured using a liquid scintillation counter. Results are presented as mean thymidine uptake+/−SEM.

### Statistical Analyses

Comparison of tumor sizes is performed using the Student's t test. Tumor incidence curves are analyzed by Wilcoxon's two-sample test.

### Expression of CD80 and/or CIITA in MCNeuA Cells

To stably express high amounts of CD80 and MHC class II molecules in MCNeuA cells, replication-defective retroviral expression plasmids are constructed encoding the cDNAs of interest and the blasticidin resistance gene. The mouse CD80, human Flag epitope-tagged CIITA, and human HA epitope-tagged h:Ub:CIITA1 open reading frames are inserted in the EcoRI site of the pWZLblast2 retroviral vector. In pWZLblast2, the genes of interest, followed by the internal ribosomal entry site (IRES) from the encephalomyocarditis virus and the blasticidin resistance gene, are transcribed from the MLV LTR (Attal et al., Genet Anal, 15:161-165, 1999; and Attal et al., Gen Expre, 8:299-309, 1999). Amphotropic retroviruses are produced and used to infect MCNeuA cells. Resistant colonies are drug-selected, expanded, and isolated by limiting dilution cloning. With each transfection, at least three independent clones are assayed for the expression of the appropriate genes. Cells expressing the highest amounts of the transduced genes are characterized further. MCNeuA cells expressing only blasticidin resistance (M), CD80 (MB), CIITA (MC), both CD80 and CIITA (MBC), h:Ub.CIITA1 (MU), or both CD80 and h:Ub.CIITA1 (MBU) are used for further experiments. Cells are examined for the expression of CIITA by Western blotting and for the expression of MHC class II by flow cytometry. All cells are expected to express high amounts of c-Neu. MB, MBC and MBU cells are also expected to express abundant CD80, whereas MC and MU cells should not express CD80. Since MC, MBC, MU and MBU cells contain CIITA, they are expected to express high amounts of MHC class II on their surface. Of note, the amounts of MHC class II expressed by these transfectants are expected to be three logs over the background fluorescence, similar to amounts of MHC class II expressed by activated B cells.

### Stimulate Lymphocyte Proliferation In Vitro

To examine functional consequences of expressing CD80 and/or CIITA in MCNeuA cells, proliferation assays are performed with non-adherent syngeneic mouse splenocytes, cocultured with M, MB, MC, MBC, MU or MBU cells in vitro. MC, MBC, MU and MBU cells are expected to increase the proliferation of lymphocytes as measured by [³H] thymidine incorporation. The coexpression of CIITA and CD80 in MBC cells is contemplated to lead to the greatest response. However, M cells even at a high responder to stimulator cell ratio (20:1) should not stimulate appreciable lymphocyte proliferation.

### Suppressed Growth of Transfected Tumor Cells in MMTV-Neu Mice

To determine whether the expression of CD80 and/or CIITA in MCNeuA cells effects tumor growth in MMTV-neu mice, groups (n=8-14) of 6-8 weeks old MMTV-neu female mice are inoculated subcutaneously with 5×10⁶ modified tumor cells and monitored for a period of 40 days. Mice in the control group are inoculated with M cells. The M cells are highly malignant with tumors expected to arise 12 days after inoculation and mice in need of sacrificing by day 40. In contrast, expression of CIITA in MC and MU cells is expected to slow tumor growth. Although the majority of the mice in this group are expected to develop tumors, these tumors are expected to be significantly smaller as compared to tumors in the MMTV-neu mice receiving M cells. The expression of either CD80 alone or CD80 and CIITA in MCNeuA cells is expected to result in complete inhibition of tumor growth in MMTV-neu mice receiving MB, MBC, or MBU cells. These experiments are repeated with two independently derived MB, MC, MBC, MU and MBU clones. The prevention of growth of mammary tumors in MMTV-neu mice is contemplated to correlate with the in vitro proliferative responses measured in vitro.

To determine if MC and MU cells continue to express MHC class II after prolonged growth in MMTV-neu mice, these tumors are explanted ˜4 weeks after the initial inoculation. The cells are analyzed for MHC class II expression by flow cytometry. Some explanted MC and MU tumor cells are expected to lose the expression of MHC class II, which is thought to reflect the ability of tumor cells bearing low levels of MHC class II to survive better in MMTV-neu mice. Although the presence of MHC class II is expected to delay the progression of MC and MU tumors, the subsequent loss of MHC class II molecules likely explains the any outgrowth of these tumors.

Immunizations with Transfected Tumor Cells Reduces the Incidence of Spontaneous Mammary Tumors in MMTV-neu Mice

To determine the effects of expressing CD80, or CD80 plus CIITA, in MCNeuA cells on the development of spontaneous mammary tumors, MMTV-neu mice are given a single injection of MB, MBC, MU or MBU cells at 6 weeks of age and followed over time for tumor growth. The injected tumor cells are expected to be rejected by the host. In addition, only a percentage of mice inoculated with MB, MBC, MU or MBU cells are expected to develop spontaneous tumors by 60 weeks of age. In contrast, control mice should begin developing spontaneous mammary tumors around 30 weeks of age and by 50 weeks of age, 100% of these mice (n=10) should have developed tumors. Immunization with MB, MBC, or MBU cells should result in a significant prolonged tumor-free period as compared to non-immunized control mice. Of note, the challenge with MBC or MBU cells rather than MB cells is expected to appreciably delay the onset of tumors.

Histologic analyses are performed on mammary tissues 35 weeks after the inoculation of MBC or MBU cells. Wild type MMTV-neu mice develop massive mammary tumors composed of poorly differentiated, densely packed tumor cells. In contrast, mice inoculated with MBC or MBU cells should not develop tumors (or will develop fewer or small tumors) and are expected to possess generally histologically normal mammary glands. The one possible exception may be the appearance of dense mononuclear infiltrates near the ducts in the mice receiving MBC or MBU cells. These infiltrates likely represent anti-tumor immune responses in the recombinant tumor cell immunized MMTV-neu mice.

### Example 3

### Tumor Cell Immunization of Human Cancer Patients

The following experiments are performed to convert tumor cells into antigen presenting cells expressing high levels of MHC II and CD80 molecules for use in the methods and compositions of the present invention for inducing a cellular immune response against tumor cells in human cancer patients (e.g., including but not limited to individuals having malignant cancer of the pancreas, gall bladder, stomach or liver).

Cancer cells obtained from a biopsy of a patient's tumor are transduced with retroviral human Ub.CIITA1 and human CD80 expression vectors in vitro, stable transfectants are selected with appropriate antibiotics. Approximately one million transduced cells are delivered to the patient's spleen via injection of the femoral vein. The remainder of the transduced cell population is frozen for subsequent administrations (e.g., to boost or otherwise enhance the patient's tumor cell reactive immune response). Prior to administration of the transduced cancer cells, patients receive a low dose of cyclophosphamide (50 mg twice a day for a week). Two days later, transduced cancer cells are administered. In some embodiments, this protocol is repeated twice as shown in Table 1.

TABLE 1
Exemplary Transfected Tumor Cell Administration Schedule

| Treatment | |
|---|---|
| Day(s) | Therapy |
| 0-7 | first chemotherapy with low dose cyclophosphamide |
| 9 | first application of transduced cancer cells |
| 14-21 | second chemotherapy with low dose cyclophosphamide |
| 23 | second application of transduced cancer cells |
| 28-35 | third chemotherapy with low dose cyclophosphamide |
| 37 | third application of transduced cancer cells |

The administration of low doses of cyclophosphamide is contemplated to decrease the number of suppressor regulatory CD4+ T cells, thereby allowing the immune system to be fully activated by the transduced cancer cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology, genetics, or related fields are intended to be within the scope of the following claims.

What is claimed is:

1. An isolated nucleic acid encoding a mammalian ubiquitin/major histocompatibility complex class II transactivator isoform 1 (Ub.CIITA1) fusion protein.

2. The nucleic acid of claim 1, wherein said Ub.CIITA1 fusion protein is the protein set forth in SEQ ID NO:2 or a transcriptionally active variant that differs from SEQ ID NO:2 by less than 1%.

3. The nucleic acid of claim 1 as set forth in SEQ ID NO:1.

4. An expression vector comprising the nucleic acid of claim 1.

5. The expression vector of claim 4, wherein said vector is a recombinant retrovirus.

6. The expression vector of claim 4, further comprising a selection marker.

7. An isolated host cell comprising the expression vector of claim 4.

8. The host cell of claim 7, wherein said host cell is a cancer cell.

9. The host cell of claim 8, further comprising an expression vector comprising a nucleic, acid encoding a mammalian co-stimulatory molecule.

10. The host cell of claim 9, wherein said co-stimulatory molecule is selected from the group consisting of CD80 (B7.1) and CD86 (B7.2).

11. The host cell of claim 10, wherein said CD80 (B7.1) is the protein set forth in SEQ ID NO:8 or a biologically active variant that differs from SEQ ID NO:8 by less than 1%.

12. The host cell of claim 10, wherein said CD86 (B7.2) is the protein set forth in SEQ ID NO:10 or a biologically active variant that differs from SEQ ID NO:10 by less than 1%.

13. A method for inducing an immune response, comprising:
a) contacting a tumor cell with an expression vector comprising a nucleic acid encoding a mammalian ubiquitin/major histocompatibility complex class II transactivator isoform 1 (Ub.CIITA1) fusion protein, under conditions suitable for producing transfected tumor cells expressing MHC class II molecules; and
b) administering said transfected tumor cells to a subject under conditions suitable for inducing an immune response against said transfected tumor cells.

14. The method of claim 13, wherein said expression vector further comprises a nucleic acid encoding a mammalian co-stimulatory molecule and said transfected tumor cells further express costimulatory molecules.

15. The method of claim 13, wherein said tumor cell is obtained from a biopsy of a tumor from said subject.

16. The method of claim 15, wherein said tumor is from an organ selected from the group consisting of breast, pancreas, gall bladder, stomach and liver.

17. The method of claim 13, wherein said immune response comprises one or more of the group consisting of a transfected tumor cell-reactive proliferative response by lymphocytes from said subject, shrinking an existing tumor of said subject, and delaying development of tumor metastases in said subject.

18. A method for inducing an immune response, comprising administering transfected tumor cells expressing MHC class II molecules to a subject under conditions suitable for inducing an immune response against said transfected tumor cells, wherein said transfected tumor cells comprise an expression vector comprising a nucleic acid encoding a mammalian ubiquitin/major histocompatibility complex class II transactivator isoform 1 (Ub.CIITA1) fusion protein.

19. The method of claim 18, wherein said expression vector further comprises a nucleic acid encoding a mammalian co-stimulatory molecule and said transfected tumor cells further express costimulatory molecules.

20. The method of claim 19, wherein said expression vector comprises at least two replication deficient retrovirus vectors.
